(19) United States
(12) Patent Application Publication
(10) Pub. No.: US 2015/0099022 A1
(43) Pub. Date: Apr. 9, 2015

(54) PHYTOECDYSONES FOR USE IN IMPROVING THE MUSCLE QUALITY OF OBESE AND/OR SARCOPENIC MAMMALS

(71) Applicants: INSTITUT BIOPHYTIS SAS, ROMAINVILLE (FR); UNIVERSITE PIERRE ET MARIE CURIE, PARIS (FR); INSTITUT NATIONAL DE LA RECHERCHE AGRONOMIQUE, PARIS (FR)

(72) Inventors: Stanislas Veillet, Savigny Sur Orge (FR); Rene Lafont, Paris (FR); Anne-Sophie Foucault, Paris (FR); Waly Dioh, Bretigny Sur Orge (FR); Annie Quignard-Boulange, Paris (FR)

(21) Appl. No.: 14/364,249

(22) PCT Filed: Dec. 13, 2012
(86) PCT No.: PCT/FR2012/052931

§ 371 Date: Aug. 25, 2014

(30) Foreign Application Priority Data
Dec. 13, 2011 (FR) 1161519

(51) Int. Cl. A61K 31/575; A61K 36/21

(52) U.S. Cl. CPC A61K 31/575; A61K 36/21
USPC 424/776; 552/551

(57) ABSTRACT

Phytoecdysones for use in improving muscle quality in obese and/or sarcopenic mammals, preferably, obese mammals subjected to a low-calorie diet. The phytoecdysones are advantageously incorporated into a food composition. The phytoecdysones can be derived from plants, such as quinoa.

### TECHNICAL DOMAIN

The invention relates to phytoecdysones provided in pure or extract form for use in improving muscle quality in mammals.

More particularly, the invention provides improvement in the muscle quality of obese mammals, and specifically obese mammals subjected to a weight-loss diet.

The invention also provides improvement in the muscle quality of sarcopenic mammals.

### THE STATE OF THE ART

Muscle quality can be defined essentially in terms of muscle strength, which is linked to the mass, the protein composition and the lipid composition of the muscle. Muscle quality in obese mammals is modified by excess intake of lipids in the muscle (Magnusson et al., 2008). Unmetabolized lipids accumulate in the muscle fibres (Goodpaster et al., 2000; Galgani et al., 2008) leading to changes in muscle metabolism, and specifically to a diminution in protein synthesis (Anderson et al., 2008; Sitnick et al., 2009) and mitochondrial activity (Kelley et al., 2002).

One method whereby an obese mammal can be enabled to lose weight and fat is to follow a low-calorie diet. Such diets are however a cause of substantial losses in muscle mass and strength (Bopp et al., 2008).

Similarly, ageing leads to pathological loss of muscle mass and strength which may in turn lead to abnormal loss of mobility and increased risk of falls (Boirie, 2008, 2009; Zamboni et al., 2008; Chérin, 2009; Rolland and Vellas, 2009; Pahor et al., 2009). Sarcopenia is the physiological phenomenon associated with ageing whereby an individual loses muscle mass, with corresponding gains in adipose mass.

Sarcopenia is a phenomenon that may lead to specific cases of obesity known as “sarcopenic obesity”.

The discovery of nutraceutical or pharmaceutical products capable of limiting loss of muscle quality in mammals suffering from obesity, sarcopenia or sarcopenic obesity in the context of nutrition-based treatment is therefore a goal pursued by numerous laboratories and manufacturers with a view to improving the care provided by nutritionists and clinicians to mammals suffering from obesity, sarcopenia or sarcopenic obesity (Lynch, 2004; Bonnefoy, 2008; Chérin, 2009; Kim et al., 2010).

Phytoecdysones are ecdysteroids of plant origin. These are natural molecules in the triterpene family and are relatively abundant in the plant kingdom, where they are present in 5% of wild plants. (Báthori and Pongrács, 2005).

As described in patent FR2924346 on behalf of the Applicant, phytoecdysones, especially 20-hydroxyecdysone, are known to reduce the increase in body fat in mammals subjected to obesifying diet.

Furthermore, these molecules have antioxidant properties (Kuzmenko et al., 2001) and no toxic effects.

### DISCLOSURE OF THE INVENTION

The inventors have discovered that ingestion of phytoecdysones, whether or not it is regular, can improve muscle quality and/or strength in obese mammals suffering from sarcopenia and/or sarcopenic obesity.

Improvement in muscle quality and/or strength is understood here to mean for example that ingestion of phytoecdysones, and 20-hydroxyecdysone in particular, can increase lean body mass in mammals subjected to an obesifying diet and that same mammal's muscle protein content. In addition, ingestion of phytoecdysones reduces loss of lean body mass in mammals subjected to a weight-loss diet. And lastly, the muscle strength of such mammals subjected to weight-loss diets, as assessed by testing, is also preserved by the ingestion of phytoecdysones.

Individuals are considered obese when their Body Mass Index (BMI) exceeds 30. In cases of sarcopenic obesity, BMI may be less than 30 due to the loss of muscle mass and a corresponding gain in adipose mass.

The invention therefore proposes to use phytoecdysones, and 20-hydroxyecdysone in particular, to improve or maintain muscle strength in obese and/or sarcopenic mammals.

A particular form of the invention uses phytoecdysones to reduce the fat content and/or increase the protein content of the muscles of obese and/or sarcopenic mammals.

A particular form of the invention uses phytoecdysones to maintain muscle strength in obese mammals subjected to a weight-loss low-calorie diet.

The phytoecdysones used can be obtained by extraction from plants containing phytoecdysones. The phytoecdysones used may also be synthesized.

The phytoecdysones should preferably be selected from 20-hydroxyecdysone, makisterone A, 24-epi-makisterone A, 24(28)-dehydro-makisterone A, 20,26-dihydroxyecdysone or mixtures of two or more of these.

The phytoecdysones used may be provided in pure form or in the form of a plant extract that has been enriched to a greater or lesser extent. The phytoecdysones used according to the invention may be provided advantageously in the form of a phytoecdysone-rich plant extract, said extract containing at least 1% by weight of phytoecdysones. Such extract should preferably contain between 1% and 7% of phytoecdysones, more preferably between 1.5% and 3% and more preferably still 2% by mass.

The plants from which the extracts are made in accordance with the invention are preferably selected from quinoa, spinach and fungi.

The phytoecdysone-rich plant extract in accordance with the invention should preferably be derived from an extract of quinoa. This is so because quinoa is an edible pseudo-cereal naturally rich in phytoecdysones (Zhu et al., 2001. Dini et al., 2005.). It is possible for example to supplement the diet with intake of phytoecdysone-rich quinoa extract by introducing that extract into foodstuffs such as dairy products or beverages, or consuming it as a dietary supplement in the form, for example, of soft capsules.

Quinoa is to date the food plant that is the richest in phytoecdysones by far. Quinoa seeds contain a combination of phytoecdysones (Zhu et al., 2001). These phytoecdysones are particularly abundant in quinoa's seed coat. For example, a 60-gram portion of quinoa seeds (dry weight) contains between 15 mg and 20 mg of 20-hydroxyecdysone.

Spinach and certain fungi may also be advantageously used to produce a plant extract rich in phytoecdysones (Findeisen, 2004).

The phytoecdysones used in accordance with the invention are advantageously presented in the form of a composition that can be administered orally.

The composition may be for example a foodstuff such as a beverage, a dairy product or other product. The composition may also of course be of medicinal type in the form of pills containing a precise dose of phytoecdysones.

### BRIEF DESCRIPTION OF THE DRAWINGS

FIG. 1: Graph representing the carcass weights of four groups of mice in an initial experimental protocol.

FIG. 2: Graph representing the triglyceride content of quadriceps muscle plotted against the diet and treatment to which the mice in the first protocol were subjected.

FIG. 3: Graph showing the protein content of quadriceps muscle plotted against the dietary regime and treatment to which the mice in the first protocol were subjected.

FIG. 4: Graph representing the gene expression levels in quadriceps muscle plotted against the dietary regime and treatment to which the mice in the first protocol were subjected.

FIG. 5: Average food intake (kcal/day) of mice according to the different treatments implemented in the first protocol.

FIG. 6: Average energy expenditure (Watt) of mice according to the different treatments implemented in the first protocol.

FIG. 7: Changes in lean body mass in obese subjects supplemented with quinoa extract (A) or placebo (B) after a low-calorie diet phase lasting six weeks, applying a second experimental protocol.

FIG. 8: Changes in measured strength using the “grip test” in obese subjects supplemented with quinoa extract (A) or placebo (B) after a low-calorie diet phase lasting six weeks, applying the second experimental protocol.

FIG. 9: The chemical formulas for the phytoecdysones present in a composition according to one embodiment of the invention.

### DETAILED DESCRIPTION

In the invention, it is proposed to provide a concentrated dose of pure phytoecdysones or using a phytoecdysone-rich plant extract to improve the muscle condition of individuals suffering from obesity, sarcopenia or sarcopenic obesity.

According to the invention, it is possible to provide this dose of phytoecdysones in the form of an extract from a plant such as quinoa, incorporated for example into a food forming part of the normal diet of an individual. Specifically, 4 grams of quinoa extract enriched with 0.5% of phytoecdysones by weight contain 20 milligrams of phytoecdysones. In order to obtain the same quantity of phytoecdysones from quinoa seeds it would be necessary to consume 50 to 100 grams of untreated seeds (Dini et al., 2005). The quinoa extract according to the invention may contain up to 50 times more phytoecdysones than the quinoa seeds from which the extract is derived.

### I—Sample Preparation Method Of Phytoecdysone-Rich Quinoa Extract (Extract A)

The method involves a sequential extraction with water, adding 500 g of quinoa seeds to 2 litres of boiling water, the whole being maintained for 5 minutes at 80° C. The water is eliminated and a second extraction is performed with 2 litres of an ethanol-water mix (1:1) applying constant agitation for 20 minutes at 80° C.

Sequential extraction of this kind eliminates saponins from the extract, these being abundant in quinoa seeds (Muir et al., 2002), and which would give a bitter taste to said extract.

The ethanol extract is filtered through Miracloth™, evaporated to dryness and taken up in 400 ml of absolute ethanol, leaving an abundant insoluble residue. The ethanol fraction is filtered or centrifuged and then dried. Chromatographic analysis (HPLC) shows that this extract contains 2±0.2% by weight of 20-hydroxyecdysone (20E).

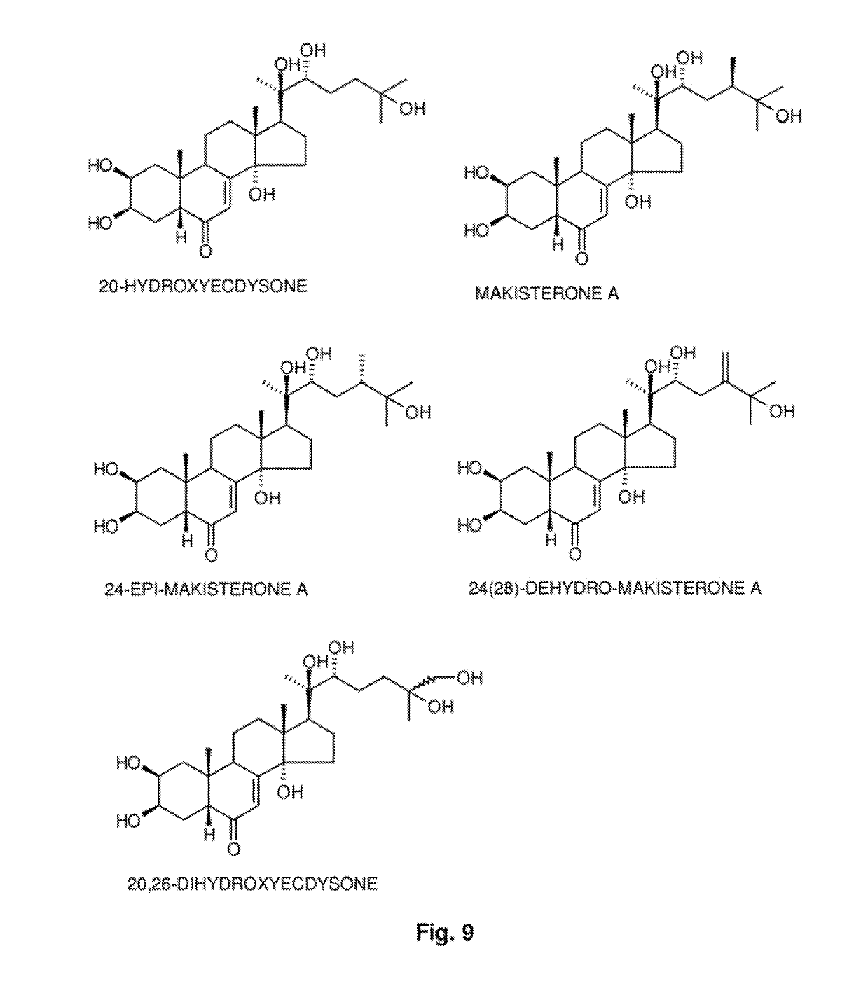

A quantity of between 150 and 200 milligrams of phytoecdysones is obtained per kilogram of treated quinoa seeds, of which 85-90% is 20-hydroxyecdysone and the remainder ecdysteroids with very similar structures such as makisterone A, 24-epi-makisterone A, 24(28)-dehydro-makisterone A or 20,26-dihydroxyecdysone. The structures of these compounds are illustrated in FIG. 9.

Most notably, an extract analogous with extract A, suitable for use in connection with the invention, is sold under the name Quinolia®.

### II—Experimental Study of the Effect of 20-Hydroxyecdysone and Extract A on the Muscle Composition of Mice Subjected to a High-Fat Diet

### Protocol

The effect of phytoecdysones is observed on mice subjected to a high-fat diet during 3 weeks.

The HF high-fat diet involved the intake of large amounts of fat in the form of lard. The mice selected for the study were male C57BL/6J mice, 6 weeks old at the start of the experiment.

Mice not subjected to a high fat diet, forming a normal diet control group, were tested in parallel.

The mice in the study were grouped according to the dietary regimes and treatments to which they were subjected: normal or control diet (LF), high-fat diet (HF), high-fat diet supplemented with quinoa extract (HFQ) and high-fat diet supplemented with pure 20-hydroxyecdysone (HF20E).

The mice were subjected to the dietary regimes detailed in Table 1 below for three weeks and the mice fed a high-fat diet were treated in parallel with pure 20E or extract A (2% 20E). The concentration of 20E was adjusted to equal 40 mg per kg of food.

In light of the average food intake of the mice, the dose of 20E administered corresponded in the two treatments to 5 mg of 20E per kg of body weight per day. The food was supplied in excess every day for both dietary regimes and all three treatments.

On average, 40 g of food was provided per cage per day or 6.5 g of food per mouse per day.

Table 1 below sets out in greater detail the composition of the diets to which mice were subjected:

TABLE 1
Composition of diets

| | |
|---|---|
| LF | HF |
| control diet | high-fat diet |

Composition (g/kg)

| | | |
|---|---|---|
| Milk proteins | 140 | 170 |
| Starch | 622.4 | 360 |
| Sucrose | 100.3 | 57 |
| Soybean oil | 40 | 40 |
| Lard | 0 | 235* |
| Mineral salts | 35 | 62.5 |
| Vitamins | 10 | 12.5 |
| Cellulose | 50 | 62.5 |
| Choline | 2.3 | 2.3 |

Energy (kcal %)

| | | |
|---|---|---|
| Proteins | 15 | 14 |
| Carbohydrates | 76 | 35 |
| Fats | 9 | 51 |

** 56% monounsaturated fatty acids, 29% saturated fatty acids and 15% polyunsaturated fatty acids (Ueda et al., 2011).

### Results

### Measurements of Animal Carcass Weights.

FIG. 1 shows the lean body mass (carcass defatted) of the animals at the end of the experiment. Administration of a high-fat diet has reduced carcass weight by 5% compared to the control group. This result is consistent with the reduction of muscle protein synthesis resulting from such a diet (Anderson et al., 2008). Supplementation with extract A has not led to a significant increase, but the 20-hydroxyecdysone has produced an increase that has allowed the mice to return virtually to the same level as the normal diet.

### Measurements of Muscle Triglyceride Content

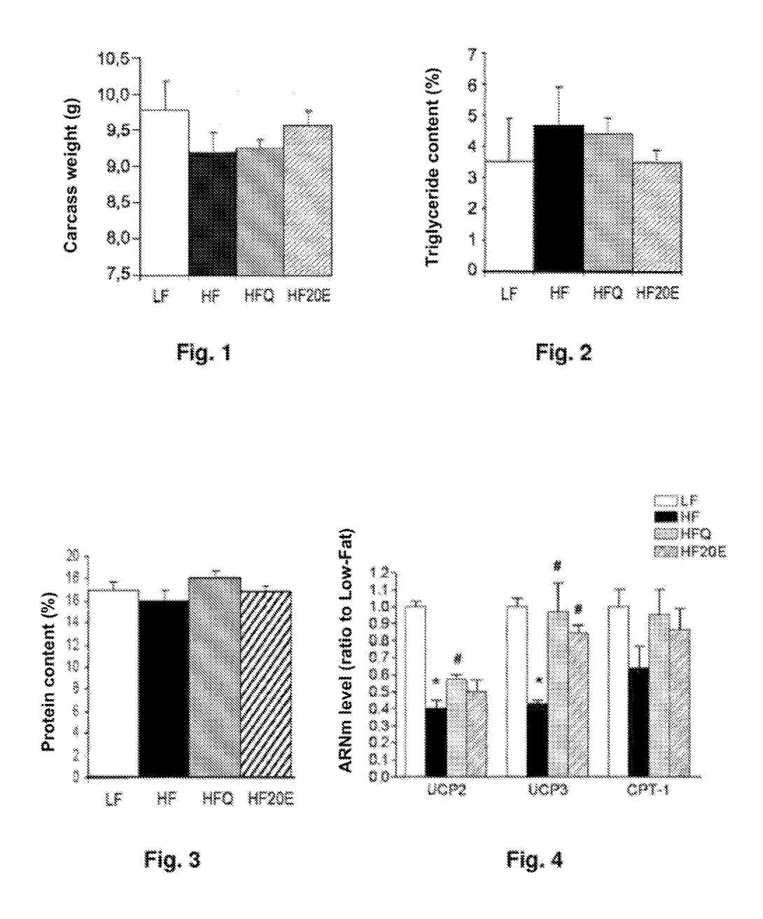

Following sacrifice, aliquots of muscle (quadriceps) were collected for analysis. FIG. 2 contains a graph plotting muscle triglyceride content against diet and associated treatment.

As expected, a trend was observed towards a higher increase in triglyceride content for the muscle in mice fed a high-fat diet compared to the control group of mice fed with the control diet (30% increase).

In the mice that had received a treatment in association with the high-fat diet, administration of pure 20E or extract A shows a trend towards lower muscle triglyceride content of 26% and 6% respectively.

### Measurements of Muscle Protein Content

FIG. 3 contains a graph plotting muscle protein content against diet and associated treatment. The high-fat diet shows a trend towards lower (−5%) muscle protein content compared to mice fed the control diet.

In the mice that had received a treatment in association with the high-fat diet, treatment with pure 20E or extract A shows a trend towards higher muscle protein content by 5% and 13%, respectively, compared to HF treatment alone.

### Measurements of Quantities of Gene Transcripts in the Muscle.

FIG. 4 contains a graph plotting quantities of gene transcripts (mRNA), as measured in the muscle, against diet and associated treatment. The quantities have been normalized with respect to the quantities measured in the muscles of mice fed the control diet.

The high fat diet produced a sharp decrease in the quantity of gene transcripts coding for uncoupling proteins UCP2 and UCP3 compared to the quantities measured in mice fed the control diet. In the mice that had received a treatment in association with the high-fat diet, administration of pure 20E resulted in an increase in the quantity of UCP3 gene transcripts and a tendency to increased quantities of UCP2 gene transcripts. In mice that had received a treatment in association with the high-fat diet, administration of extract A led to an increase in the quantity of UCP2 and UCP3 gene transcripts. The high-fat diet led to a decrease in the quantity of gene transcripts coding for CPT-1 intracellular fatty acid transporter relative to the amount measured in mice fed the control diet. Treatment with pure 20E and extract A therefore tends to restore transcript levels to those seen for the control diet. These changes are consistent with an improvement in muscle oxidative capacity due to treatment with pure 20E and extract A.

### Energy Balance

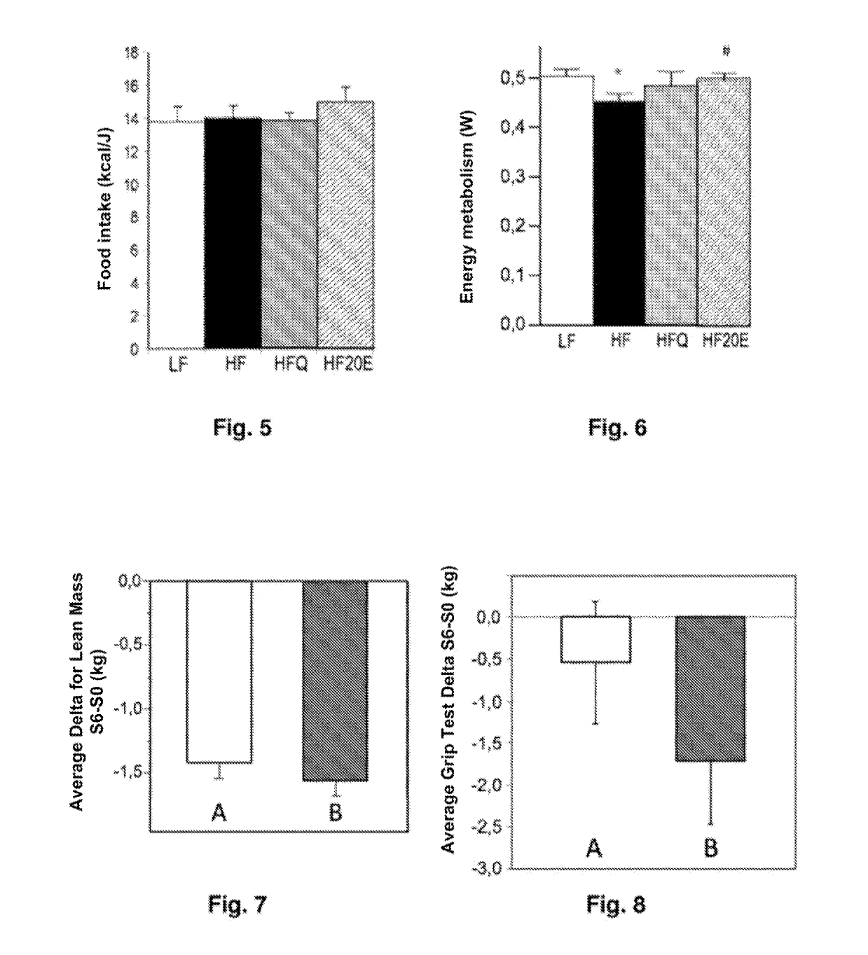

The animals fed the high-fat diet consumed a quantity of food providing them with the same amount of energy (kcal) as animals fed the standard diet (FIG. 5). This is also true for the animals receiving extract A or pure 20E. Conversely, the energy expenditure of the latter was higher (9%) than that of the animals fed the high-fat diet alone (FIG. 6). This difference, although small, has important implications, because its effect was cumulative over the duration of the experiment.

### Conclusion of the Experiments on Mice

The administration of pure 20E, like that of extract A, prevents the lipid deposition and protein loss in muscle induced by a high-fat, lard-based diet. Both treatments promote the metabolism of fatty acids taken up in excessive amounts in muscle due to the administration of the high-fat diet.

The increased energy expenditure combined with constant food intake may explain the observed differences in the accumulation of fat. This increased energy expenditure was not due to increased locomotor activity (as measured in metabolic cages); it appears therefore to be due to increased thermogenesis.

### III—Clinical Double-Blind Study of the Effects of Extract a on Obese Individuals Subjected to a Low-Calorie Diet for 6 weeks

### Protocol

The effect of extract A was studied on protection of lean mass during a low-calorie diet. The effect of extract A was studied in a double-blind clinical study involving obese subjects following a low-calorie diet for 6 weeks. Protection of lean mass was assessed by measuring muscle strength using a “grip test” and by estimating lean body mass in a DXA scan analysis of body composition.

The muscle strength and lean mass data are estimated values. To take into account differences in the duration of the low-calorie diet phase, the grip test and lean body mass data were initially calculated per day actually completed before being multiplied by the 42 days corresponding to the average duration of the low-calorie diet phase undergone by the volunteers.

### Measurement of the Loss of Lean Body Mass During the Low-Calorie Diet Phase

The effect of extract A on protection of lean body mass was studied during a low-calorie diet period. The product leads to a slight tendency to greater protection of lean body mass compared with the placebo (FIG. 7).

It is likely that the metabolic constraints of a stringent diet outweigh all other considerations and indeed, in studies conducted outside any such diet period, the “anabolic” effects of 20-hydroxyecdysone were significantly enhanced by supplementary intake of proteins (Simakin et al., 1988).

Changes in grip test data during the low-calorie diet phase

The effect of administration of extract A on muscle quality in obese subjects subjected to a low-calorie diet was studied. The measured changes in grip test results after 6 weeks of diet (FIG. 8) evidence greater protection of muscle strength in subjects ingesting extract A supplements (−0.55 kg) than in those who received a placebo (−1, 70 kg).

### Conclusions

Administration of extract A provides obese subjects with enhanced protection of lean body mass as is shown by the DXA scan analysis, with a trend towards lower loss in the case of extract A compared to the placebo. Muscle quality is also better protected by administration of extract A, the loss being smaller compared to the group receiving the placebo.

### REFERENCES

Anderson S R, Gilge D A, Steiber A L, Previs S F. 2008. Diet-induced obesity alters protein synthesis: tissue-specific effects in fasted versus fed mice. Metabolism 7(3): 347-54.
Chermnykh N S, Shimanovsky N L, Shutko G V, Syrov V N. 1988. Effects of methandrostenolone and ecdysterone on physical endurance of animals and protein metabolism in the skeletal muscles. Farmakologiya i Toksikologiya 6: 57-62.
Foucault A S, Lafont R, Dioh W, Fromentin G, Veillet S, Tomé D, Quignard-Boulangé A. 2008. Effets d'un extrait de quinoa enrichi en 20-hydroxyecdysone sur l'adiposité dans le cadre du syndrome métabolique. Nutrition Clinique et Metabolism 22 (supp1.1), S103.
Gadzhieva R M, Portugalov S N, Paniushkin V V, Kondrat'eva 11. 1995. A comparative study of the anabolic action of ecdysten, leveton and Prime Plus, preparations of plant origin. Eksp Klin Farmakologiya 58(5): 46-48.
Garcia-Martinez C, Sibille B, Solanes G, Darimont C, Mace K, Villarroya F, Gomez-Foix AM.2001. Overexpression of UCP3 in cultured human muscle lowers mitochondrial membrane potential, raises ATP/ADP ratio, and favors fatty acid vs. glucose oxidation. FASEB J. 15: 2033-2035.
Gong D W, He Y, Karas M, Reitman M. 2000. Uncoupling protein-3 is a mediator of 20 thermogenesis regulated by thyroid hormone, [33-adrenergic agonists, and leptin. J. Biol. Chem. 272(39): 24129-24132.
Gorelick-Feldman J, MacLean D, Hic N, Poulev A, Lila MA, Raskin I. 2008. Phytoecdysteroids increase protein synthesis in skeletal muscle cells. J. Agric. Food Chem. 56: 3532-3537.

Li B, Nolte LA, Ju JS, et al. (2000) Skeletal muscle respiratory uncoupling prevents diet-induced obesity and insulin résistance in mice. Nature Medicine 6: 1115-1120. Schrauwen P, Hesselink M (2002) Review. UCP2 and UCP3 in muscle controlling body metabolism. J. Exp. Biol. 205: 2275-2285.
Seidlova-Wuttke D, Ehrhardt C, Wuttke W. 2010. Metabolic effects of 20-OH-ecdysone in 30 ovariectomized rats. J. Steroid Biochem. Mol. Biol. 119: 121-126.
Simakin SYu, Panyushkin V V, Portugalov S N, Kostina L V, Martisorov EG. 1988. Combined application of preparation Ecdysten and product Bodrost during training in cyclic sports. Sports Science Bulletin N°2, 29-31.
Tôth N, Szabô A, Kacsala P, Heger J, Zàdor E. 2008. 20-Hydroxyecdysone increases fiber35 size in a muscle-specific fashion in rat. Phytomedicine 15: 691-698.
Ueda Y, Wang MF, Wei AV, Sarukura N, Sakai T, Hsu TF. 2011. Effect of Dietary Lipids on Longevity and Memory in the SAMP8 Mice. J. Nutr. Sci. Vitaminol. (Tokyo) 57(1): 36-41.
Veillet S, Lafont R. (2009) Use of phytoecdysones for the preparation of a composition foracting on metabolic syndrome. Patent WO 2009071804, application WO 2008- FR52088, application FR 2007-59478 US 2011/0033561 A1.
Wang S, Subramaniam A, Cawthorne M A, Clapham J C (2003) Increased fatty acid oxidation in transgenic mice over-expressing UCP3 in skeletal muscle. Diabetes, Obesity and Metabolism 5: 295-301.

1. Phytoecdysones for use in improving or maintaining muscle strength in at least one of the following: obese or sarcopenic mammals.

2. Phytoecdysones as claimed in claim 1 for use in reducing at least one of following: fat content or increasing protein content of muscle in mammals suffering from obesity or sarcopenic obesity.

3. Phytoecdysones according to claim 1, for use in maintenance of muscle strength in obese mammals subjected to a weight-loss low-calorie diet.

4. Phytoecdysones according to claim 1 consisting of 20-hydroxyecdysone.

5. Phytoecdysones according to claim 1, provided in the form of a plant extract enriched with one or more phytoecdysones.

6. Phytoecdysones as claimed in claim 5, wherein the extract comprises at least 1% by weight of phytoecdysones.

7. Phytoecdysones according to claim 5, wherein the plant extract is derived from quinoa.

8. Phytoecdysones according to claim 1 incorporated in a composition suitable for oral administration.
